(19) United States
(12) Patent Application Publication
(10) Pub. No.: US 2025/0213109 A1
(43) Pub. Date: Jul. 3, 2025

(54) REFRACTIVE DIOPTRIC POWER DETERMINATION METHOD

(71) Applicant: TOKAI OPTICAL CO., LTD., AICHI (JP)

(72) Inventors: Hitoshi MIURA, Aichi (JP); Eiji SUZUKI, Aichi (JP)

(21) Appl. No.: 18/852,226

(22) PCT Filed: Jan. 18, 2023
(86) PCT No.: PCT/JP2023/001260

§ 371 Date: Sep. 27, 2024

(30) Foreign Application Priority Data
Mar. 31, 2022 (JP) 2022-058460

(51) Int. Cl. A61B 3/032; A61B 3/00

(52) U.S. Cl. CPC A61B 3/032; A61B 3/0025; A61B 3/0033

(57) ABSTRACT

[Abstract] A dioptric power determination method is provided that is capable of accurately calculating not only the lens diopter power but also both the astigmatic power and the astigmatic axis according to a convenient subjective method and that is capable of reflecting a halfway test result on the final dioptric power without fear of the occurrence of overcorrection.

[Solution] The method includes the steps of setting a target value of the eyesight in a state in which a subject has been subjected to refractive-error correction; allowing the subject to watch a plurality of optotypes facing various different directions in a state of wearing a test lens or in a naked-eye state while regarding the target value as target eyesight; asking the subject to answer the direction of the optotype; if a result in which a correct answer and an incorrect answer, or a correct answer and an indistinguishable answer, or a correct answer, an incorrect answer, and an indistinguishable answer coexist is obtained, estimating a dioptric power by which the subject visually perceives the optotype with a fixed predetermined probability in all directions in a circumferential direction of the optotype corresponding to the target eyesight on a basis of a relationship between a response and a dioptric power corresponding to the response; and determining the dioptric power of the ophthalmic lens of the subject on a basis of an estimation result obtained by estimating the dioptric power.

### TECHNICAL FIELD

The present invention relates to a dioptric power determination method for determining the dioptric power of an ophthalmic lens when vision correction is made by the ophthalmic lens.

### BACKGROUND ART

Generally speaking, an eyesight test for a user (i.e., subject) is performed when ophthalmic lenses (eyeglass lenses, contact lenses) are newly prescribed for the user.

### CITATION LIST

### Patent Documents

Patent Document 1: Japanese Published Unexamined Patent Application No. 2020-199250
Patent Document 2: Japanese Translation of PCT International Application No. 2020-518858

### SUMMARY OF INVENTION

### Technical Problem

However, there are some problems in performing the eyesight test and determining the dioptric power of the lens.

First, if the astigmatic power is included in the dioptric power in a subjective eyesight test in which a subject makes a determination of whether visible or invisible, many procedures are required to determine an astigmatic power (C dioptric power) and an astigmatic axis (Ax), and there is a fear that the determination method will become troublesome and complex, and burdens will be imposed both on the subject and on a person who measures the eyesight of the subject.

Also, according to an objective test in which an autorefractometer is used, it is possible to determine the dioptric power of the subject in a few seconds. However, this objective test has the problem of eye accommodation (which is called mechanical near-sightedness) that occurs when a person peers into the autorefractometer or a similar device. Also, there are cases in which eyestrain is saved if the degree of correction is set at a low level so that slightly poorer eyesight than the best eyesight can be obtained by an eyesight correction when eyeglasses or contact lenses are worn. On the other hand, the autorefractometer is a mechanism that measures a dioptric power to obtain the best eyesight in principle. Further disadvantageously, there is a concern that overcorrection will be caused if the dioptric power to obtain the best eyesight is measured in a state of mechanical near-sightedness (accommodation phenomenon caused by the act of peering into). Therefore, in many cases, it is considered improper that the dioptric power determined by the autorefractometer is prescribed as the dioptric power of eyeglasses or contact lenses without changes.

As a means of solving these problems, a technique for automatizing the refraction measurement or the eyesight test has been proposed as in, for example, Patent Document 1. Patent Document 1 discloses a method in which automatization is achieved by reading a numerical value, such as a dioptric power engraved on a trial lens, by use of a camera and by executing the voice recognition of a subject's response. This technique has been developed to fulfill a desire to reduce the effort involved in measurement chiefly in an eyesight test and a desire to perform the eyesight test as conveniently as possible in a short time, and yet the steps of this technique are executed by use of a very large-scale device, and therefore this technique is not convenient in practice, and it is difficult to introduce such a large-scale device.

Also, Patent Document 2 discloses a method for accurately determining an astigmatic correction value by decomposing the astigmatic power and the axis into J00/J45 according to a Jackson cross cylinder method. However, even if this method is employed, all of the problems described above are not solved. Only information about two finally-compared conditions that are trial results obtained through a trial and error process is used to determine a final dioptric power, and therefore information about the trial and error process performed to reach a final conclusion is not necessarily reflected, and an entirely satisfactory conclusion cannot be obtained. Also, a subject is allowed to determine the fact that “visual perceptions under two conditions are nearly equal to each other,” and therefore the result is affected by the subjectivity of the subject.

Also, disadvantageously, the subject becomes tired, or the state of the visual perception is altered during the eyesight test as a problem that is common to both Patent Document 1 and Patent Document 2. In view of these respects, some people have the notion that the employment of the final result that is a result of the trial and error process is irrational as a test result used to determine the final dioptric power.

Therefore, a dioptric power determination method has been desired which is capable of accurately calculating not only the lens diopter power (S dioptric power) but also both the astigmatic power (C dioptric power) and the astigmatic axis (Ax) according to a convenient subjective method and which is capable of reflecting a halfway test result on the final dioptric power without fear of the occurrence of overcorrection.

### Solution to Problem

To solve the aforementioned problems, means 1 provides a dioptric power determination method for determining a dioptric power of an ophthalmic lens when eyesight is corrected by the ophthalmic lens, the method including the steps of setting a target value of the eyesight in a state in which a subject has been subjected to refractive-error correction by the ophthalmic lens; allowing the subject to watch a plurality of optotypes facing various different directions in a state of wearing a test lens or in a naked-eye state while regarding the target value as target eyesight; asking the subject to answer the direction of the optotype; if a result in which a correct answer and an incorrect answer, or a correct answer and an indistinguishable answer, or a correct answer, an incorrect answer, and an indistinguishable answer coexist is obtained, estimating a dioptric power by which the subject visually perceives the optotype with a fixed predetermined probability in all directions in a circumferential direction of the optotype corresponding to the target eyesight on a basis of a relationship between a response and a dioptric power corresponding to the response; and determining the dioptric power of the ophthalmic lens of the subject on a basis of an estimation result obtained by estimating the dioptric power.

If estimation is performed so as to equalize the probability of being visually perceivable with target eyesight in all directions in the circumferential direction of the optotype corresponding to target eyesight in this way, it is possible to determine the dioptric power of an ophthalmic lens desired by a subject on the basis of a calculation result obtained by a convenient technique of calculation, thus making it possible to provide an ophthalmic lens that is accurate and that does not have the concern of causing overcorrection with respect to not only the lens diopter power (S dioptric power) but also the astigmatic power (C dioptric power) and the astigmatic axis (Ax).

The “ophthalmic lens” is merely required to be a lens having a dioptric power determined by an eyesight test, and is, for example, an eyeglass lens and a contact lens. The “dioptric power” is a dioptric power for an eyeglass lens or for a contact lens appropriate to vision correction, and concretely denotes a set of values of “S dioptric power, C dioptric power, and Astigmatic axis” for lens order.

Preferably, the “test lens” is, for example, a trial lens that is capable of being detachably attached to a temporary frame (trial frame) so as to have various dioptric powers by lens replacement, and the “test lens” may be an eyeglass lens having a clear dioptric power attached to an eyeglasses frame wearable as eyeglasses. Also, the “test lens” includes a lens having no dioptric power. Also, from the viewpoint of acquiring data, the test lens may be not only a trial lens but also an eyeglass lens worn by a subject at the present time. Also, data tested by the naked-eye may be included in data tested by use of the test lens during data acquisition process.

An operator who has a role to indicate an optotype as in a conventional manner as a main person performing a subject's eyesight test while using the “test lens” is not necessarily required. For example, an optotype chart may be shown on a screen of, for example, a monitor, or the test may be performed by using a technique of virtual reality with a VR device. For example, a case is acceptable in which the test is performed by software of a computer. Also, an operator is not necessarily required to exist with a subject in the same space even if the operator exists, and the operator may issue a command from a remote place as a person working remotely.

The reason for allowing a subject to watch an optotype in a “naked-eye state” is that an eyesight test is first performed with present naked vision in many cases if the subject is a person who uses an ophthalmic lens for the first time, and a base is created without test lenses if the naked vision is not too far apart from the target eyesight. Data tested by use of the test lens may be included in data tested by the naked-eye during a data acquisition process.

Means 1 is the idea of acquiring the dioptric power of the ophthalmic lens in subject's target eyesight by performing estimation on the basis of a result of a subjective eyesight test. Therefore, the subject is allowed to repeatedly watch optotypes in practice, and, as a result, data is acquired. The data is combination data of both an answer and the dioptric power corresponding to the answer. Although a plurality of pieces of data are needed, it is recommended to acquire as many pieces of data as possible in order to improve the accuracy of an estimated value. The subjective eyesight test is a test in which the subject voluntarily answers the direction of an optotype indicated by the operator in a state of watching the optotype only with a so-called single eye.

Also, a response is required to be made so that a correct answer and an incorrect answer, or a correct answer and an indistinguishable answer, or a correct answer, an incorrect answer, and an indistinguishable answer coexist. In other words, the present invention is to estimate the dioptric power of an ophthalmic lens in subject's target eyesight while using pieces of data indicating that an optotype is visible or invisible depending on the size or the direction of the optotype. Therefore, a case (correct answer) in which the subject always answers “visible” to optotypes that have been indicated or, on the contrary, a case in which the subject always answers “invisible” (incorrect answer) to the optotypes is not assumed here.

Therefore, the subject is prohibited to have such a biased visual perception when the subject is allowed to wear a test lens and is allowed to repeatedly watch an optotype. In other words, when a basic dioptric power is determined, there is a case in which the eyesight leads to extreme overcorrection that is extremely far from subject's corrected eyesight or a case in which the subject is allowed to wear a lens having a very weak dioptric power regardless of the fact that the subject has excessive myopia. However, even if there is a case in which such an extreme visual perception is created at the beginning, a trial and error process is applied to the eyesight test so that the eyesight gradually approaches target eyesight while changing a trial lens, generally speaking, if subject's naked vision is unclear, and therefore the response comes to include a correct answer, an incorrect answer, and an indistinguishable answer sooner or later. The indistinguishable answer denotes a case in which the subject cannot determine a direction in which the optotype faces, and answers “indistinguishable.”

Also, from the step of estimating a dioptric power by which the subject visually perceives the optotype with a fixed predetermined probability in all directions in a circumferential direction of the optotype corresponding to the target eyesight, the fact that the optotype is visually perceivable with a fixed predetermined probability in all directions denotes that the refractive-error correction of the subject has been appropriately performed. In other words, this is a dioptric power in which the rate of a correct answer and an incorrect answer, or a correct answer and an indistinguishable answer, or a correct answer, an incorrect answer, and an indistinguishable answer coincides in value with the fixed predetermined probability.

It is safe to say that “estimating a dioptric power by which the subject visually perceives the optotype with a fixed predetermined probability in all directions” is “assuming a probability function formula in which the value of a correct-answer probability reaches a predetermined probability and estimating a dioptric power that maximizes the likelihood obtained by applying a corresponding test result (optotype direction, optotype size, correct or incorrect of response) based on the probability function formula.” It is recommended to use, for example, a logistic function formula as the probability function formula. “Dioptric power that seems to become a predetermined correct-answer probability in all directions” is estimated because of estimation. “All directions” denotes “all” in 360 degrees, and this does not denote that data is acquired by testing concerning “all directions,” of course. Only a direction concerning which an eyesight test is performed is used for an actual calculation, and the accuracy becomes higher in proportion to the number of directions concerning which a test is performed, and yet a calculation is performed so as to maximize the likelihood by use of the function formula, and therefore there is no need to perform a test concerning all directions. Strictly, “dioptric power that seems to become a predetermined correct-answer probability in all directions” is estimated.

The fixed probability is to be predetermined, and the weight may be appropriately changed. For example, when the weight of a correct answer and the weight of an incorrect answer are equalized with each other (for example, 1), and when the weight of an indistinguishable answer is set at 0.5, each weight is calculated as 0.5 on the assumption that there is one correct answer and there is one incorrect answer.

Optotypes having various different sizes and directions are repeatedly watched, and many responses are acquired (i.e., a data amount is increased), and, as a result, the fixed predetermined probability in all directions in the circumferential direction of target eyesight is approached, and, based on this result, a dioptric power is estimated.

As a concrete method for estimation, it is recommended to find a likelihood and to calculate the dioptric power of an ophthalmic lens in target eyesight by optimization calculation while performing estimation according to the maximum-likelihood method. It is recommended to find a likelihood and to apply the formula of an appropriate likelihood-representing probability function on the basis of the likelihood, and it is recommended to perform estimation according to that formula. It is possible to formulate the formula of a probability function according to, for example, a logistic regression formula, or a probit regression formula that uses a cumulative distribution function of normal distribution, or a similar formula. The probability function formula, the maximum-likelihood method, and the optimization calculation will be described later.

Also, in means 2, the dioptric power of the test lens is changed, and the subject is allowed to wear the test lens, and subject is allowed to repeatedly watch the optotype in order to mix a correct answer and an incorrect answer, or a correct answer and an indistinguishable answer, or a correct answer, an incorrect answer, and an indistinguishable answer together when the subject is allowed to watch the optotype in a state in which the subject is wearing the test lens.

Although data is able to be acquired by a single test lens, it is possible to acquire more various kinds of data by changing the dioptric power of the test lens and by allowing the subject to wear the test lens, and it is possible to improve the accuracy of calculation of an estimation numerical value.

Also, in means 3, when the subject is allowed to watch the optotype in a state in which the subject is wearing the test lens, a dioptric power of the test lens to mix a correct answer and an incorrect answer together, or mix a correct answer and an indistinguishable answer together, or mix a correct answer, an incorrect answer, and an indistinguishable answer together is set at a dioptric power of an eyeglass lens regularly used by the subject or at a dioptric power close to the dioptric power of the eyeglass lens.

The dioptric power of a test lens serving as the base of a test is set as a standard of the dioptric power of an eyeglass lens that is regularly used by the subject, and, as a result, it is possible to prevent extreme data out of subject's corrected eyesight from coexisting, and it is possible to improve the accuracy of calculation of an estimation numerical value in a state in which the number of times of a test is small.

It is recommended to set the “dioptric power close to the dioptric power” at a dioptric power slightly closer to the plus side, or, on the contrary, at a dioptric power slightly closer to the minus side than, for example, the dioptric power of the eyeglass lens regularly used by the subject. Also, it is recommended to employ a dioptric power brought close to a spherical power by weakening the astigmatism. In other words, the dioptric power is a dioptric power obtained by slightly changing the dioptric power of the eyeglass lens regularly used by the subject.

Also, in means 4, the test lens has a same dioptric power in each watching and in each answering, and the subject is allowed to wear this test lens and is allowed to repeatedly watch the optotype.

The subject is allowed to watch the optotype in this way, and hence the subject is enabled to be subjected to a test without changing the test lens, and it contributes to a convenient, swift eyesight test.

Also, in means 5, when the subject is allowed to wear the test lens, the subject wears the test lens having a dioptric power differing in accordance with a test situation, and repeatedly watches the optotype.

This watching manner makes it possible to acquire more various kinds of data, and makes it possible to improve the accuracy of calculation of an estimation numerical value.

The test situation is to, when the subject is allowed to watch the optotype and obtains a response, change the test lens depending on the contents of the response. For example, it denotes a case in which a correct answer is made concerning all optotypes displayed on the eyesight-test chart because of an overcorrected test lens or, on the contrary, a case in which an incorrect answer or an indistinguishable answer is made concerning all optotypes.

Also, in means 6, the optotype watched by the subject is a plurality of optotypes that differ in size including the optotype corresponding to the target eyesight.

This makes it possible to acquire more various kinds of data, and makes it possible to improve the accuracy of an estimation numerical value.

Also, in means 7, the optotype is displayed by an eyesight-test chart so that different-sized optotypes are visually perceivable at a glance.

This makes it possible to visually perceive different-sized optotypes at a glance. Additionally, it is possible to comprehend the outline of visible sizes and invisible sizes of a group of optotypes group at a glance, and therefore it is easy to sensuously understand determination of a first one of the visually perceivable sizes. As a possible situation, the chart may be arranged in front of the subject as a table in practice, or may be watched in a device, such as a horopter device, as an image through an optical system. Preferably, many kinds of different directions are prepared as a pattern of directions of a group of different-sized optotypes arranged at the chart.

Also, in means 8, a direction of the optotype in a group of the optotypes displayed on the eyesight-test chart consists of two kinds of directions, i.e., a certain direction and a direction 180 degrees opposite to the certain direction.

In other words, a group of the optotypes displayed on the eyesight-test chart do not face various directions, and is composed of the optotypes having only two directions one of which is a certain direction and the other one of which is a direction 180 degrees opposite to the certain direction. As a result, the subject has no need to anticipate optotypes having many directions, thus enabling the subject to make a fast judgement without great perplexity over how to make a response.

Also, in means 9, the number of kinds of the directions of the optotype is six to sixteen.

The reason is that, if the number of kinds of the directions of the optotype is too small, the number of kinds of to-be-acquired data becomes small, and the accuracy of estimation becomes low. On the other hand, if there are many kinds in the direction of an optotype, a slight directional difference is not easily understood, and extra labor hours will be imposed on the subject, excluding how to allow the subject to watch two kind of directions 180 degrees opposite to each other at a time. Additionally, if the optotype is invisible, many incorrect answers will occur, and the rate at which data of “accidental hit” randomly generated with low frequency affects an estimation result of the dioptric power becomes high, and the accuracy becomes low in that sense. If how to allow the subject to watch two kind of directions is employed, the “accidental hit” occurs with a probability of ½, and therefore the effects on an estimation result are averaged. Preferably, angular distances between the directions of optotypes are equal to each other.

Also, in means 10, an eyesight value according to a size of the optotype is a log MAR form.

log MAR is in relationship with log(1/decimal eyesight). For example, decimal eyesight 1.0 corresponds to log MAR eyesight 0.0. If log MAR is used, numerical values are arranged at equal intervals in comparison with decimal eyesight, and therefore, if log MAR optotypes are used in a test, pieces of data arranged at equal intervals on a graph are acquired, and therefore high efficiency is achieved.

Also, in means 11, the optotype is a Landolt ring.

The Landolt ring is the most general as an optotype, and the use of the Landolt ring is the most proper from the matching with a conventional eyesight test. However, a figure other than the Landolt ring may be used as an optotype.

Also, in means 12, the estimation is performed by optimization calculation according to a maximum-likelihood method.

In other words, a likelihood may be found in the estimation calculation, and the value of a probability function formula representing the likelihood is maximized. This is performed according to a maximum-likelihood method (method of estimating a parameter on the assumption that assuming that the most plausible result has been acquired). In this means, the estimation is performed by optimization calculation, and a well-known steepest descent method, quasi-Newton's method, conjugate gradient method, etc., may be employed as an optimizing method.

Also, in means 13, calculation by which a likelihood is calculated in the optimization calculation may be performed by logistic regression, and may be estimated on a basis of the likelihood.

The logistic regression is one of the techniques for establishing a formula of a likelihood function in the maximum-likelihood method, and can be formulated as an approximation formula by which a calculation is easily performed. This logistic regression makes it possible to perform a simpler calculation than, for example, probit regression to which a cumulative distribution function of normal distribution is applied.

The present invention is not limited to configurations described in the following embodiments. It is recommended to configure each embodiment and components of each embodiment by arbitrarily selecting and combining those components together. Additionally, it is recommended to arbitrarily combine and configure arbitrary components of each embodiment or of modifications and arbitrary components described in the “means to solve the invention” or components that materialize arbitrary components described in the “means to solve the invention” together. With respect to these configurations, the applicant has will to acquire the right in the amendment or division of the present application.

### Effects of Invention

According to the present invention, it is possible to determine the dioptric power of an ophthalmic lens desired by a subject on the basis of a calculation result obtained by a convenient technique of calculation, thus making it possible to provide an ophthalmic lens that is accurate and that does not have the concern of causing overcorrection with respect to not only the lens diopter power (S dioptric power) but also the astigmatic power (C dioptric power) and the astigmatic axis (Ax).

### BRIEF DESCRIPTION OF DRAWINGS

FIG. 1 A block diagram that describes a peripheral device to perform the calculation of a dioptric power determination method in an embodiment of the present invention.

FIG. 2 A descriptive view that describes an eyesight chart used to determine the dioptric power when an eyesight test is started in the embodiment of the present invention.

FIG. 3 A descriptive view that describes an eyesight chart used to perform a first test of the eyesight test in the same embodiment.

FIG. 4 A descriptive view that describes an eyesight chart used to perform a second test of the eyesight test in the same embodiment.

FIG. 5 A descriptive view that describes an eyesight chart used to perform a third test of the eyesight test in the same embodiment.

FIG. 6 A descriptive view that describes an eyesight chart used to perform a fourth test of the eyesight test in the same embodiment.

FIG. 7 A graph of a logistic curve that represents a relationship between log MAR eyesight and a correct-answer rate of a subject based on data obtained in the same embodiment.

FIG. 8 A graph in which where a logistic curve related to an estimated dioptric power is added to the logistic curve that represents a relationship between the log MAR eyesight and the correct-answer rate of the subject based on data obtained in the same embodiment.

FIG. 9 A descriptive view that describes a relationship between Landolt rings that face twelve directions and number symbols of a clock face that are used in another embodiment.

FIG. 10 A descriptive view that describes directions of Landolt rings and arrangement directions that are used in an eyesight chart in another embodiment.

### DESCRIPTION OF EMBODIMENTS

An example of embodiments of a dioptric power determination method of the present invention will be hereinafter described.

First, a description will be given of a schematic configuration of an example of a peripheral device that calculates a probability function according to logistic regression and that makes an optimization calculation according to a maximum-likelihood method in the embodiment of the present invention.

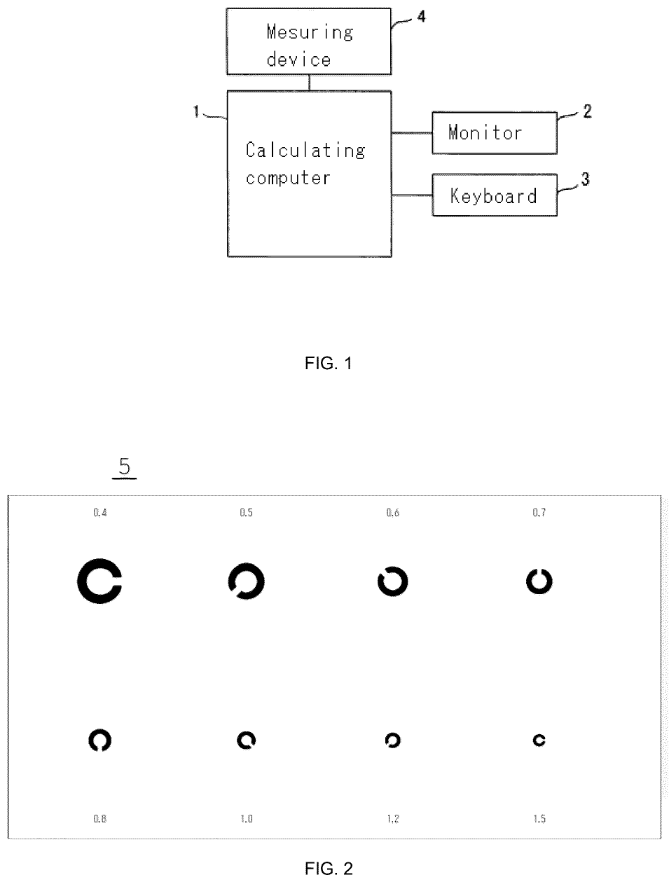

A monitor 2 and a keyboard 3 are connected to a calculation computer 1 as shown in FIG. 1. The keyboard 3 serves as an input means for inputting numerical values in this embodiment.

A printer, an output means that transfers data to other devices, and the like, besides the monitor 2, can be mentioned as an output means. Also, a means that inputs data transferred from another LAN-connected computer or from another device, such as a data-storage device, can be mentioned as the input means, besides the keyboard 3.

The calculation computer 1 is composed of peripheral devices, such as CPU (central processing unit), ROM, RAM, etc., as an electrical configuration. The CPU performs logistic regression based on a data group obtained by an eyesight test in accordance with a calculation program stored in the ROM, and performs a calculation by which the likelihood is maximized. Thereafter, a dioptric power to determine the dioptric power of an ophthalmic lens of a subject is determined on the basis of numerical values obtained by the calculation.

Next, an example of a process performed until the dioptric power is determined will be described in detail in the embodiment.

### A. Concerning Data Acquisition in Eyesight Test

Here, an example in which data about a case in which the number of directions of an optotype is finally sixteen is acquired will be described.

a. Acquisition of Basic Dioptric Power

This stage is a stage at which a basic dioptric power is roughly determined. Therefore, objective measurement may be performed with a device, such as an autorefractometer even if a trial and error process is not performed while actually using a trial lens, and the dioptric power may be set to be the same as that of eyeglasses wearing at the present time. At this stage, a roughly-determined dioptric power may be employed without problem, and therefore, if an astigmatic power and an astigmatic axis are unknown, the astigmatic power is not necessarily required. Based on the dioptric power acquired in this way, a trial lens by which an optotype of 1.0 is visually perceptible is set in a temporary frame, and is worn by the subject. If the subject has an astigmatic power, this astigmatic power may be added to a spherical power.

b. Determination of Startup Dioptric Power

The dioptric power of a trial lens determined in the aforementioned item a. is set as a temporary startup dioptric power, and an eyesight test is started while using an eyesight chart 5 of FIG. 1 on which a plurality of Landolt rings that are optotypes are displayed. Thereafter, a trial lens that makes the Landolt ring of 1.0 visible is found by the eyesight test. Data acquired in the eyesight test from the stage of b. is used in the calculation mentioned below.

At this time, it is recommended to limit the number of directions of the Landolt rings to not sixteen but eight or four. The reason is that fewer directions of the Landolt rings enable the subject to confidently make a determination of whether visible or invisible. The eyesight chart 5 of FIG. 2 shows Landolt rings that are eight in direction. The eyesight chart 5 of FIG. 2 may be actually arranged in front of the subject, or may be displayed by a horopter. In the eyesight chart 5 of FIG. 2, eight Landolt rings that differ in size from each other are arranged in a two-tiered manner, and four among the eight Landolt rings are arranged at substantially equal intervals in each tier. The Landolt rings are arranged in order of size that becomes smaller from the left side toward the right side, and the Landolt ring placed in the upper tier at the left end is the largest, and the Landolt ring placed in the lower tier at the right end is the smallest. A decimal eyesight value is shown in each of the Landolt rings. The Landolt rings of the eyesight chart 5 have eight directions of equal angular intervals of, for example, 0 degrees in the right, 180 degrees in the left, 90 degrees in the upper, 270 degrees in the lower, 45 degrees in the upper-right, 225 degrees in the lower-left, 135 degrees in the upper-left, and 315 degrees in the lower-right when the rightward horizontal direction is defined as 0 degrees.

Here, the fact that the subject is able to “visually perceive” an optotype denotes a case in which the direction of the optotype (Landolt ring) is able to be correctly answered. In this embodiment, when an eyesight test is performed, a period of time may be set, and an answer may be made within, for example, three seconds. Also, conditions, such as the condition that an optotype is presented only during, for example, three seconds, may be attached. Also, the fact that the subject is unable to “visually perceive” an optotype denotes a case in which the direction of the optotype is unable to be correctly answered. In addition, cases are included in which a wrong direction is answered, or in which the subject answers “indistinguishable,” or in which an answer cannot be made within the time limit.

In this state, a trial is conducted in which the subject is allowed to make an answer while replacing a trial lens set in the temporary frame with another trial lens, and the value of a spherical-power lens is adjusted so that the optotype of 1.0 becomes visible. If a horopter is used, the dioptric power of the trial lens is adjusted by an operator who operates the horopter.

As a concrete eyesight-test technique, the dioptric power of the trial lens is adjusted (the kind of the lens is replaced with another), and, as a result, a dioptric power is selected by which the four Landolt rings in the upper tier are visible and by which the smallest one among the four Landolt rings in the lower tier is invisible. The three Landolt rings in order from the left among the four Landolt rings in the lower tier may be visible or may be invisible. There is a range in the condition that the Landolt ring of decimal eyesight 0.7 is visible and that the Landolt ring of 1.5 is invisible, and therefore, in many cases, it is possible to reasonably determine the dioptric power of the lens. The distance from the subject's eye to the Landolt ring depends on the eyesight chart 5, and, generally speaking, the distance is often set at five meters, and there is a case in which measurement is performed with a distance of three meters.

c. First Test and Acquisition of First Dioptric Power

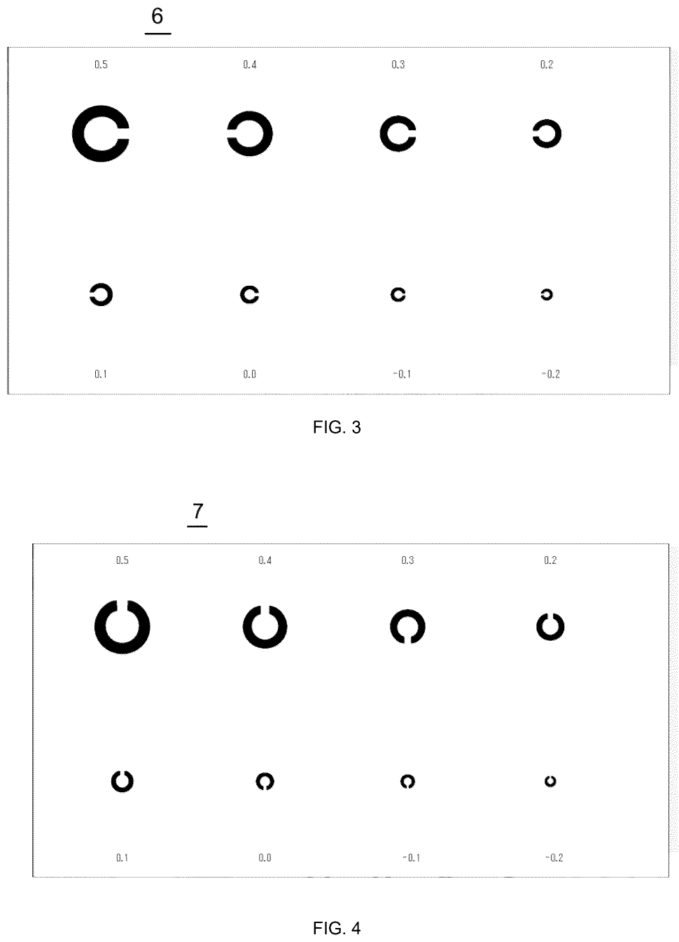

In the first test, an eyesight test is performed on the basis of FIG. 3. In the eyesight chart 6 of FIG. 3, eight Landolt rings that differ in size from each other are arranged in a two-tiered manner, and four among the eight Landolt rings are arranged at substantially equal intervals in each tier. The Landolt rings are arranged in order of size that becomes smaller from the left side toward the right side, and the Landolt ring placed in the upper tier at the left end is the largest, and the Landolt ring placed in the lower tier at the right end is the smallest. In each of the Landolt rings, the eyesight value is shown in a log MAR form. Decimal eyesight 1.0 corresponds to log MAR eyesight 0.0.

The Landolt rings of the eyesight chart 7 have only two directions in 180-degrees correspondence, i.e., 0 degrees in the right and 180 degrees in the left when the rightward horizontal direction is defined as 0 degrees.

A concrete eyesight test according to a technique is performed so that a state in which the Landolt ring of 0.2 is visible and in which the Landolt ring of −0.2 is invisible is brought about by adjusting the dioptric power of the trial lens (by replacing the kind of the lens with another). If the Landolt ring of 0.2 is invisible, the minus of the spherical power lens is strengthened. For example, the dioptric power of the trial lens is changed into a minus value 0.25 D by 0.25 D. If the Landolt ring of −0.2 is visible, the minus of the spherical power lens is weakened. For example, the dioptric power of the trial lens is changed into a plus value 0.25 D by 0.25 D. To prevent the subject from remembering the direction of the Landolt ring when the test is performed while allowing the subject to watch the monitor 2, it is recommended to reset the direction of the Landolt ring and change the display of the monitor 2 whenever the lens is replaced with another. The dioptric power adjusted and acquired in this way is defined as a “first dioptric power.”

When the adjustment is completed, the subject is again asked to answer the direction of each of all Landolt rings of the eyesight chart 7 in a state of wearing the trial lens having the first dioptric power, and the items of correct answer ∘, incorrect answer x, and indistinguishable answer Δ are recorded. In these items, “∘: correct answer” includes a case in which the subject perceives the optotype visually correctly and a case in which the subject makes a correct answer contingently, and “x: incorrect answer” is a case in which the subject perceives the optotype visually incorrectly, and “Δ: indistinguishable answer” is a case in which the subject feels that “the optotype is invisible.”

When the subject makes an answer by guesswork even if the subject feels that “the optotype is invisible,” the subject's answer becomes not Δ but ∘ or x with a probability of ½. Even in that case, the dioptric power estimated after all becomes substantially the same.

Eight data are recorded at a time in the eyesight chart 7, and, here, it is presumed that the answers become almost correct with respect to four optotypes (Landolt rings) of from 0.5 to 0.2. With respect to the Landolt ring of −0.2, presumably, there is a case in which the subject has a consciousness of being “indistinguishable,” and there is a case in which the subject makes an incorrect answer as a result.

In this step of the first test, the items of correct answer ∘, incorrect answer x, and indistinguishable answer Δ may be recorded even in a stage in which the “first dioptric power” whose adjustment has not yet been completed is adjusted, and the result obtained here may be used. The reason is that more data that are used for calculation enhance the possibility that good results will be obtained.

d. Second Test and Acquisition of Second Dioptric Power

In the second test, an eyesight test is performed on the basis of FIG. 4. In the eyesight chart 7 of FIG. 4, eight Landolt rings that differ in size from each other are arranged in a two-tiered manner, and four among the eight Landolt rings are arranged at substantially equal intervals in each tier. The Landolt rings are arranged in order of size that becomes smaller from the left side toward the right side, and the Landolt ring placed in the upper tier at the left end is the largest, and the Landolt ring placed in the lower tier at the right end is the smallest. In each of the Landolt rings, the eyesight value is shown in a log MAR form. Decimal eyesight 1.0 corresponds to log MAR eyesight 0.0.

The Landolt rings of the eyesight chart 8 have only two directions in 180-degrees correspondence, i.e., 90 degrees in the upper and 270 degrees in the lower when the rightward horizontal direction is defined as 0 degrees.

A concrete eyesight test according to a technique is performed so that a state in which the Landolt ring of 0.2 is visible and in which the Landolt ring of −0.2 is invisible is brought about by adjusting the dioptric power of the trial lens (by replacing the kind of the lens with another). At this time, if it is possible to change only the dioptric power in the horizontal direction without changing the dioptric power in the vertical direction in a to-be-replaced lens, it is desirable to do so. For example, if only a spherical power lens is set in a temporary frame, it is easy to do so. The reason is that it is possible to maintain the dioptric power in the vertical direction by adding an astigmatic power lens so as to strengthen the minus in the horizontal direction or by weakening the minus of a spherical power lens and adding a minus astigmatic power lens in the vertical direction.

If the astigmatic axis is 180 degrees or 90 degrees, it is possible to make adaptation as the eyesight test even if the spherical power lens and the astigmatic power lens have already been piled up together as a trial lens. If the astigmatic axis is diagonal, a change in dioptric power in the horizontal direction is unavoidable when the lens is replaced with another, and yet it is possible to maintain the dioptric power in the horizontal direction according to a method in which an SC axis is decomposed into mdp, J00, and J45 by use of a Jackson cross cylinder, and then mdp and J00 are adjusted, and a returning operation again to the SC axis is performed.

$$mdp = S dioptric power + 0.5 \times C dioptric power$$

$${J}_{00} = - 0.5 \times C dioptric power \times cos ( 2 \times astigmatic axis \times π / 180 )$$

$${J}_{45} = - 0.5 \times C dioptric power \times sin ( 2 \times astigmatic axis \times π / 180 )$$

The multiplication by π and the division by 180 are the conversion from degrees to radians.

The dioptric power adjusted and acquired in this way is defined as a “second dioptric power.”

When the adjustment is completed, the subject is again asked to answer the direction of each of all Landolt rings of the eyesight chart 8 in a state of wearing the trial lens having the second dioptric power in the same way as the first dioptric power of b., and the items of correct answer ∘, incorrect answer x, and indistinguishable answer Δ are recorded. The items of correct answer ∘, incorrect answer x, and indistinguishable answer Δ may be recorded even in the dioptric power whose adjustment has not yet been completed, and the result obtained here may be used. Also, in order to increase data, the eyesight chart 6 of FIG. 3 may be again displayed in this state, and the Landolt ring of 0 degrees in the right and the Landolt ring of 180 degrees in the left may be displayed, and the items of correct answer ∘, incorrect answer x, and indistinguishable answer Δ may be recorded.

e. Adjustment of Dioptric Power to Acquisition of Third Dioptric Power

Based on data acquired so far, the dioptric power of the lens is determined so that the correct answer rate of the optotype of 0.0 becomes approximately 75% in both horizontal and vertical directions. There are a sufficient number of data about the optotypes in the horizontal direction and in the vertical direction, and therefore logistic regression is temporarily conducted at this stage, and the dioptric power of the subject's ophthalmic lens is calculated. Of course, subsequent data are added, and, as a result, a more highly-accurate dioptric power is expected to be calculated. A technique for determining the dioptric power of the subject's ophthalmic lens by estimating the dioptric power will be described in “B. Dioptric-Power Estimation Method” mentioned below.

Also, this stage is an intermediate stage of all data acquisition, and therefore the dioptric power of the subject's ophthalmic lens may be supposed by a rough guess without daringly calculating the dioptric power of the subject's ophthalmic lens by logistic regression. For example, if a correct answer has never been made with respect to the Landolt ring of 0.0 in the Landolt rings facing the horizontal direction and if a correct answer has been made only in the Landolt ring of 0.0 or only in the Landolt ring of 0.1, the perpendicular dioptric power is set at the minus side by 0.25 D in comparison with that condition. Alternatively, if data acquired in the Landolt rings facing the horizontal direction include only data in which a correct answer is made up to the Landolt ring of −0.1, a possible technique is that the perpendicular dioptric power is set at the plus side by 0.25 D in comparison with a condition under which a test is conducted in a state in which the perpendicular dioptric power is set at the maximal plus side.

The dioptric power calculated by performing logistic regression in this way or the dioptric power adjusted by a rough guess is defined as a “third dioptric power.”

Preferably, the third dioptric power is a state in which “the optotype of 0.2 is visible, and the optotype of −0.2 is invisible” in both the horizontal and vertical directions. However, if such a state cannot be created, the fact that the optotype of 0.2 is invisible is not a serious problem, and therefore adjustment is made so that the optotype of −0.2 is invisible. In order to deal with this state in a stage in which the third dioptric power is adjusted, twelve optotypes of, for example, from 0.7 to −0.4 may be presented, and a test may be performed in a state in which “the optotype of 0.3 is visible, and the optotype of −0.3 is invisible.”

f. Third Test

The first test and the second test primarily aim to determine the dioptric power of the subject's trial lens through a trial and error process (although acquired data are used), and yet this third test aims to acquire many data used to calculate a more highly-accurate dioptric power by use of the third dioptric power.

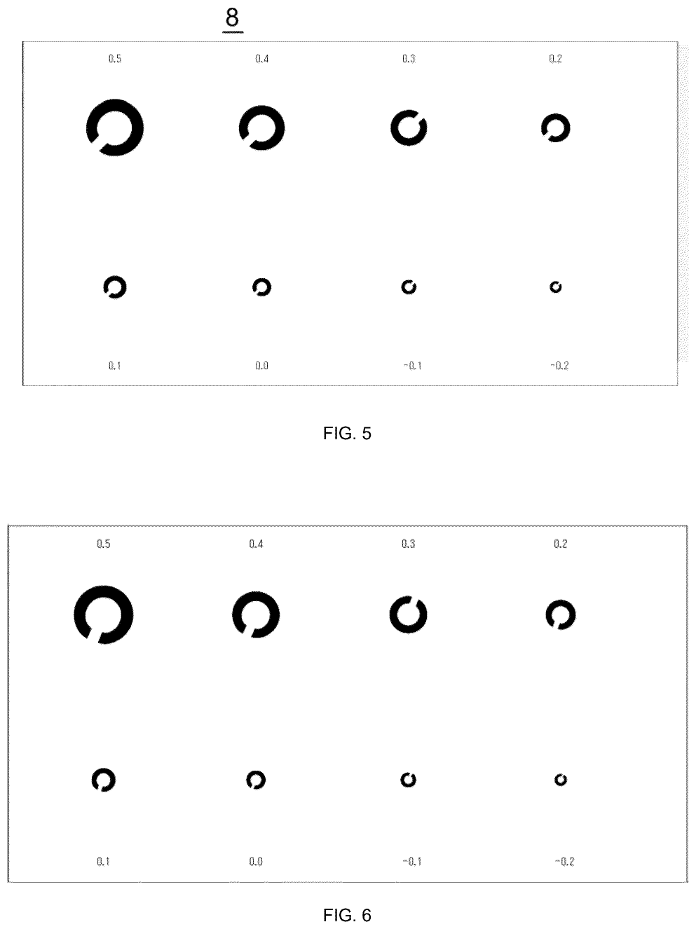

An eyesight chart that has only Landolt rings facing the two directions in 180-degrees correspondence as have been performed in “c. First Test” and in “d. Second Test” is used in the eyesight test in the third test. In the third test, the subject is asked to answer the directions of all Landolt rings, and the items of correct answer ∘, incorrect answer x, and indistinguishable answer Δ are recorded in each eyesight chart consisting of a combination of Landolt rings in directions facing each other among the four conditions of 1) 0 degrees in the right and 180 degrees in the left, 2) 90 degrees in the upper and 270 degrees in the lower, 3) 45 degrees in the upper-right and 225 degrees in the lower-left, and 4) 135 degrees in the upper-left and 315 degrees in the lower-right. The eyesight chart 8 of FIG. 5 shows “case 3)” among the cases of the Landolt rings.

g. Estimation of Dioptric Power

Based on data acquired so far, logistic regression is performed, and the dioptric power of the lens is determined so that the correct answer rate of the direction of the Landolt ring of 0.0 becomes approximately 75% in all directions. A technique for determining the dioptric power of the subject's ophthalmic lens by performing the logistic regression will be described in “B. Dioptric-Power Estimation Method” mentioned below.

If the amount of data is not adequate at this time, it is possible to perform a calculation in a state in which some to-be-estimated parameters are fixed at a predetermined value (a value determined on the basis of many subjects). The dioptric power estimated in this way is defined as a “fourth dioptric power.”

h. Fourth Test to Estimation of Dioptric Power

The process subsequent to this is performed to further improve the accuracy of the dioptric power acquired as a result. Therefore, this step is not indispensable.

A lens having the fourth dioptric power estimated in “g. Estimation of Dioptric Power” is set in the temporary frame, and a test is performed under each of new four conditions, and logistic regression is performed by use of results obtained before. The logistic regression is described in “B. Dioptric-Power Estimation Method.” An eyesight chart consisting of a combination of Landolt rings in directions facing each other as follows is used under the new four conditions, and the subject is asked to answer the directions of all Landolt rings, and the items of correct answer ∘, incorrect answer x, and indistinguishable answer Δ are recorded.

The direction of this angle cannot be easily expressed, and it is troublesome to answer the direction of the optotype in oral, and therefore it is recommended for the subject to make an answer in the form of, for example, the right, the left, the upper, and the lower after understanding that it is slightly oblique.

The new four conditions are 22.5 degrees in the upper-right and 202.5 degrees in the lower-left, 67.5 degrees in the upper-right and 247.5 degrees in the lower-left, 112.5 degrees in the upper-left and 292.5 degrees in the lower-right, and 157.5 degrees in the upper-left and 337.5 degrees in the lower-right. The inclusion of these degrees makes it possible to acquire data at equal angles concerning sixteen directions in total. As an example, an eyesight chart 9 of Landolt rings facing each other in a 180-degrees direction of 22.5 degrees in the upper-right and 202.5 degrees in the lower-left and is shown as in FIG. 6.

Estimation at this stage performs logistic regression while including these results in data accumulated until “f. Third Test” that has already been conducted. The logistic regression will be described in “B. Dioptric-Power Estimation Method.”

At this time, estimation may be formed by enlarging the weight of newer data. The reason is that new data are data acquired by conducting a test in a state in which the dioptric power has approached a dioptric power that is finally determined.

i. Variation

In the aforementioned steps, the step of “b. Determination of Startup Dioptric Power” is not indispensable. The step of “c. First Test and Acquisition of First Dioptric Power” may be performed from the beginning. Also, the aim is to raise the accuracy by acquiring many data, and therefore all of the steps of from b. to f. are not necessarily needed. For example, the step of “g. Estimation of Dioptric Power” may be performed without adjusting arbitrary dioptric powers of the first, second, and third dioptric powers in the steps of c., d., and e. Only the data about the oblique direction may be acquired in “f. Third Test.”

Also, in order to further raise the accuracy, the dioptric power may be further estimated, and the fifth dioptric power may be found. The value measured by the autorefractometer, or the previous test result, or the dioptric power of the eyeglass lens being worn at the present time may be used as the third dioptric power in the step of e. In that case, it is recommended to repeatedly perform the third test and estimation of the dioptric power and the fourth test and estimation of the dioptric power in order to acquire a highly accurate result.

The third dioptric power acquired in “f. Third Test” and in “g. Estimation of Dioptric Power” and a test result of the fourth dioptric power may be collectively used for estimation of the fifth dioptric power.

### B. Dioptric-Power Estimation Method

An example in which estimation is concretely made while using the data acquired in the step of A. will be described.

First, calculation by which the subject's eyesight is estimated while using the data acquired above in the step of 1. will be described. An estimate value to be finally found is the dioptric power of the subject's ophthalmic lens, and it is also possible to estimate the eyesight by use of the data acquired above, and therefore a case in which the eyesight is first estimated before estimating the dioptric power of the subject's ophthalmic lens will be described.

### 1. Estimation of Subject's Eyesight

Based on data of the eyesight test acquired in the step of A., logistic regression is performed, and a condition that maximizes a logistic-regression formula value according to the maximum-likelihood method is calculated, and an estimate value of the subject's eyesight is calculated from the condition. More specifically, a calculation is performed in which a value of a result obtained by adding values obtained by taking the logarithm of the logistic-regression formula value together with respect to all data is maximized as in Formula 2.

Data of a single eyesight test is composed of i) Dioptric power of lens worn by subject, ii) Eyesight value, iii) Direction of Landolt ring, and iv) Any one of correct answer ∘, incorrect answer x, and indistinguishable answer Δ. In estimation of the subject's eyesight of 1., a calculation is made by use of ii) and iv) that are taken from the aforementioned data.

“Correct answer,” “Incorrect answer,” and “Indistinguishable answer” are considered as follows.

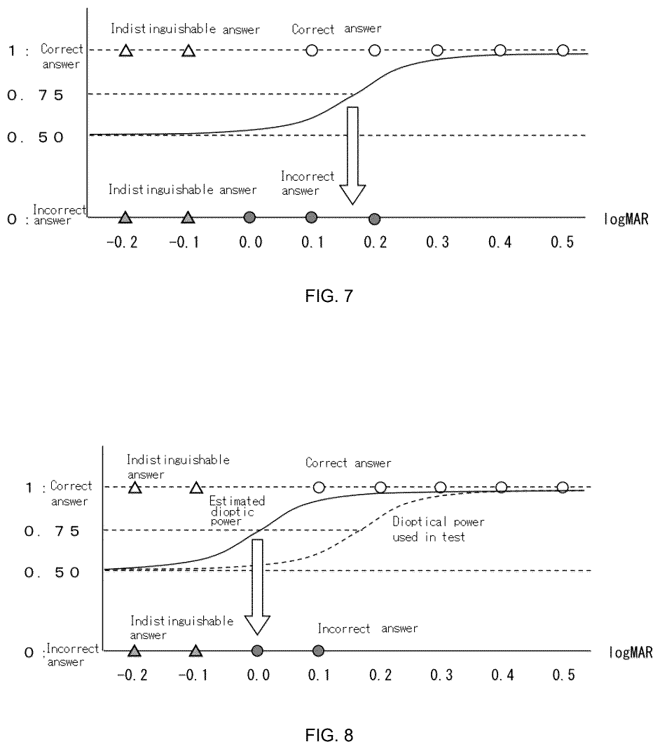

When an optotype (Landolt ring) of an eyesight is correctly seen at a rate of a half in a state in which the subject is wearing a lens having a certain dioptric power, ½ of the invisible remaining half is also right, and therefore the eyesight of a correct-answer rate of ¾ (0.75) is set as an estimate value. A logistic curve is determined as in FIG. 7 under a condition that maximizes the likelihood of acquired data. In this logistic curve, a value obtained by subtracting a distance (whose value is from 0 to 1) in the longitudinal direction between the curve and data from 1 is “Likelihood.” In other words, likelihood becomes large if the curve passes close by data. A value obtained by multiplying all likelihoods relative to each data together is likelihood relative to all data. Therefore, a condition that maximizes a value that takes the logarithm of likelihood of all data shown in Formula 1 mentioned below is calculated. Formula 1 is a logistic function formula. The logistic function formula is a formula in which 0.75 is an estimate value as described above. Formula 2 is a function formula to make an optimization calculation by applying a logistic function formula. In Formula 2, each data is a sum of values each of which takes the logarithm of likelihood of each data, and is called a logarithmic likelihood sum. If the subject correctly answers the direction of the Landolt ring, the value of g is used without changes, and if the subject incorrectly answers the direction of the Landolt ring, the value of 1-g is used. If the subject makes an “indistinguishable answer,” the resulting data of the “indistinguishable answer” is treated as two data of both a correct answer and an incorrect answer when calculated, and each weight is set as half (0.5). In other words, a result value in which g is used without changes and a result value in which 1-g is used are added together in sigma, and the weight used when added together is set as 0.5. Thereafter, an optimization calculation that maximizes a value that thus takes the logarithm of likelihood is performed. This calculation is performed by the calculation computer 1 mentioned above. The optimization calculation is performed according to a well-known optimization calculation method. An example of the optimization calculation will be described later in “2. Estimation of Dioptric Power of Subject's Ophthalmic Lens.”

$$g ( i ) = \frac{0.5}{1 + exp \{ - b \cdot ( {M}_{i} - ME ) \}} + 0.5 \qquad [ Formula 1 ]$$

$$f ( a , b , ME ) = {\sum }_{i = 1}^{n} {w}_{i} \cdot ln ( Correct answer g : Incorrect answer 1 - g ) \qquad [ Formula 2 ]$$

T: Dioptric power; In the case of astigmatic power, dioptric power in a direction relative to an optotype

Mi: Log MAR-value of an optotype corresponding to each data

Wi: Weight of each data. Data of an unknown result is adjusted to be half the value

ME: Estimate value of log MAR eyesight

b: Parameter having a positive value which is estimated simultaneously with ME. This may be predetermined by data of many subjects. There is incorrect-answer data of log MAR 0.2 in the drawing, and, if there is no incorrect-answer data thereof, estimation cannot be made, and therefore a predetermined value is used.

### 2. Estimation of Dioptric Power of Subject's Ophthalmic Lens

Here, a calculation is made while using all of the acquired data i) to iv). Additionally, Formula 3 that is a logistic function formula is used in the same way as in “1. Estimation of Subject's Eyesight.” The basic concept of Formula 3 is as follows.

A dioptric power Rθ in a direction is determined by an estimated dioptric power (SC axis). A logarithmic likelihood sum concerning a test result of a Landolt ring (whose direction is a direction perpendicular to its dioptric-power direction) corresponding to its dioptric-power direction.

Let it be supposed that, at this time, a logistic curve corresponding to an estimated dioptric power passes through the point of (0.0, 0.75) as shown in FIG. 8. In other words, the dioptric power is supposed as a dioptric power that realizes the log MAR eyesight of 0.0. If the dioptric power Tθi that has been actually used for the test differs from Rθ, a difference therebetween is reflected in the likelihood calculation. This leads to estimation in which the curve passes through the point of (0.0, 0.75) by using the term of −a (Rθ−Tθi) in exp of Formula 4. In a case in the eyesight acquired by dioptric power Tθi is weak with respect to, for example, 0.0 that is a target value, the optotype of 0.0 comes to be visible if the dioptric power of the lens is closer to the minus side, and therefore Rθ should be a more minus value than Tθi, and, in that case, the value of −a (Rθ−Tθi) in exp is required to become plus. In other words, the dotted logistic curve is shifted to the left-hand solid line.

The likelihood corresponding to each data is calculated by the optimization calculation according to Formula 4 in such a shifted state, and its logarithm is taken, and weight is multiplied, and values of parameters mdp, J00, and J45 that maximize a result sum are calculated by the optimization calculation. Formula 4 shows sixteen directions θ, an i-th test in the θ direction, and the sum total of combinations of eight optotypes (Landolt rings) j in the i-th test. The optimization calculation uses, for example, the steepest descent method mentioned above. The dioptric power Rθ and the values of parameters a and b are estimated by performing the optimization calculation. Already-known values may be applied as the parameters a and b.

In Formula 4, each data is the sum of values that take the logarithm of each likelihood of each data, and is called a logarithmic likelihood sum. If the subject correctly answers the direction of the Landolt ring, the value of g is used without changes, and if the subject incorrectly answers the direction of the Landolt ring, the value of 1-g is used, in the same way as in Formula 2. If the subject makes an “indistinguishable answer,” the resulting data of the “indistinguishable answer” is treated as two data of both a correct answer and an incorrect answer when calculated, and each weight is set as half (0.5). In other words, a result value in which g is used without changes and a result value in which 1-g is used are added together in sigma, and the weight used when added together is set as 0.5. Thereafter, an optimization calculation that maximizes a value that thus takes the logarithm of likelihood is performed. This calculation is performed by the calculation computer 1 mentioned above.

Unlike “1. Estimation of Subject's Eyesight” mentioned above, the value of ME is fixed at 0.0 in Formula 4. The reason is that the dioptric power of a lens in which 0.0 is acquired as a value of log MAR is determined. Also, the logistic function of Formula 3 has a term of the dioptric power, which is not included in the logistic function of Formula 1. The reason is that the data of correct answer, incorrect answer, and indistinguishable answer of a result obtained by wearing a lens used for a test is used in Formula 1, and therefore information on the dioptric power is not needed, and yet, lenses having various dioptric powers are used in a test performed to acquire data that serves as a base of Formula 3, and there is a need to reflect a difference between the dioptric power of a lens (which is the dioptric power of a lens used in a test and which has a direction-dependant value according to θ) and the dioptric power Rθ to be estimated.

Next, an actual example of an optimization calculation in which Formula 3 is applied to Formula 4 will be described in detail on the basis of Tables 1A to 1C and Table 2 shown below.

Tables 1A to 1C are calculation results obtained by using Formula 3 and Formula 4 on the basis of all data acquired at a stage at which sixteen tests have been performed. The estimation dioptric power Rθ, the function g (θ, i, j), and the logarithmic likelihood are calculated on the basis of data acquired by performing a test every four times on the basis of newly acquired data, and are appropriately updated. Here, as an example, a way according to which the estimation dioptric power Rθ, the function g (θ, i, j), and the logarithmic likelihood are calculated is shown on the basis of an S dioptric power, a C dioptric power, and a numerical value of the astigmatic axis AX that were estimated at a stage at which the test was performed sixteen times.

For example, in an eighth test, a calculation will be described concerning a case in which the answer was that the log MAR eyesight is 0.2 and concerning a case in which the answer was the log MAR eyesight is 0.1. In these answers, 0.2 is a correct answer ∘, and 0.1 is an incorrect answer x.

From Table 2, the parameters a and b are a: 5.14, and b: 21.46.

Also, the estimation dioptric power Rθ is −1.06 (D), and the test dioptric power Tθi is −0.75 (D).

Mθij is 0.2 and 0.1. ME is 0.0 (target eyesight).

The estimation dioptric power Rθ of −0.106 (D) is calculated as follows.

Estimated JCC is (−0.81, 0.11, −0.25) as shown in Table 2.

The value of JCC is variously changed so as to maximize likelihood in the optimization calculation. Therefore, the SC axis is calculated based on the value of JCC that has been changed, and the following calculation is further performed based on that, and the estimation dioptric power Rθ is calculated. When JCC is calculated from the SC axis, an opposite calculation is performed, and the S dioptric power, the C axis dioptric power, and the astigmatic axis are calculated on the basis of the value of JCC. The reason for performing a calculation on the basis of the value of JCC is that its calculation is more continuous than the SC axis in which a change in value is made from 180 degrees to 0 degrees, and is hence advantageous to the optimization calculation.

$$S dioptric power + C dioptric power \cdot sin 2 ( π \cdot ( astigmatic axis - dioptric power direction ) / 180 ) = 0.54 - 0.54 \cdot sin 2 ( π \cdot ( 147 - 67.5 ) / 180 ) = 0.106$$

The value of function g is calculated by substituting these values for the expression of function g (θ, i, j), i.e., for Formula 3. Here, the values are 0.969 and 0.818, respectively, as shown in Table 1B.

The concrete numerical value of function g (θ, i, j) is determined, and therefore the following calculation is performed while applying its numerical value according to Formula 4.

$$ln ( 0.969 ) = - 0 \text{.032}$$

$$ln ( 1 - 0.818 ) = - 1.703$$

This result is listed in Table 1C corresponding thereto.

This calculation is performed while being updated and while including the past data every four times of the test in this embodiment.

$$g ( θ , i , j ) = \frac{0.5}{1 + exp \{ - a ( {R}_{θ} - {T}_{θ i} ) - b \cdot ( {M}_{θ ij} - ME ) \}} + 0.5 \qquad [ Formula 3 ]$$

$$f ( mdp , J 00 , J 45 , a , b ) = {\sum }_{θ} {\sum }_{i = 1}^{n ( θ )} {\sum }_{j = 1}^{m ( θ , i )} {w}_{θ ij} \cdot ln ( Correct answer g : Incorrect answer 1 - g ) \qquad [ Formula 4 ]$$

Rθ: Dioptric power in the direction of θ, and an estimated value. That is determined by estimate values of mdp, J00, and J45. The estimate values of mdp, J00, and J45 are transformed into S dioptric power, C dioptric power, and astigmatic axis, and are calculated according to S dioptric power+C dioptric power×sin² ((astigmatic axis−θ)/180×π). θ is the direction of dioptric power. The dioptric power changes in accordance with the square of a trigonometric function by an angle.

Tθi: Dioptric power in the direction of θ, and is determined by the dioptric power of a lens used in an i-th test. This is calculated according to S dioptric power+C dioptric power×sin² ((astigmatic axis−θ)/180×π).

ME: Value of log MAR eyesight that is a target value. This is set at 0.0 in the embodiment.

Mθij: Log MAR value of optotype corresponding to each data. j is 1 to 8 correspondingly to the optotype in an i-th test in the direction of θ.

wθij: Weight of each data. The data of an unknown result is adjusted to half the value. The weight of a result obtained by performing a test while wearing a lens whose dioptric power is away from a final dioptric power may be reduced.

n(θ): Index to make a calculation when there is data about the fact that a test was performed a plurality of times in the direction of θ. This is an integer that represents times.

m(θ, i): Index to make a calculation without recording all of eight every time although the assumed value is 8.

a, b: Positive parameters that are simultaneously estimated. These may be predetermined on the basis of data of many subjects. These may be not constants but functions of dioptric power and others (e.g., pupil diameter, age, and the like).

Next, in a case in which the number of directions of the Landolt ring is sixteen, the eyesight test has been actually performed once by once, i.e., 16 times in total while using an eyesight chart on which eight Landolt rings are shown as mentioned above. In other words, an example in which 128 (=16×8) pieces of test data have been acquired and a calculation has been performed is shown in the following tables. In Table 1A to Table 1C, results of sixteen tests performed and obtained with respect to a subject are each shown in the lateral direction, and values obtained by performing an optimization calculation on the basis of the concrete sixteen tests are shown (L eye in this example). Although Table 1A to Table 1C should be actually displayed in a continuous form, these tables are displayed in a divided form because of these long lengths. This example is an example in which the value of log MAR of the lens is 0.0, and parameters a and b are simultaneously estimated. Table 2 shows estimation results. Also, Table 3 shows results of estimation calculations of a four-times test, an eight-times test, and a twelve-times test among tests performed sixteen times in total, and shows results of estimation calculations performed sixteen times when parameters a and b are fixed. The numeric precision becomes higher in proportion to the number of pieces of data, and the accuracy becomes higher in proportion to times the test is performed.

If the number of pieces of data is small, the estimation of parameters a and b is liable to become unstable. Therefore, here, parameters a and b are simultaneously estimated only in a case in which data of sixteen tests are used. Presumably, the values of a and b depend on a subject, or depend on a dioptric power. Therefore, it is recommended to beforehand find average values of a and b, for example, on the basis of many subjects, and is recommended to perform estimation in a state in which the values of a and b are fixed if the number of pieces of test data is small (in a case in which the test is performed up to four times or up to eight times).

TABLE 1A

| | SC axis | | | Direction | | | | | | | | |
|---|---|---|---|---|---|---|---|---|---|---|---|---|
| | S | C | | of | | | | | | | | |
| Times | dioptric | dioptric | | optotype | LogMAR eyesight | | | | | | | |
| of test | power | power | Axis | (degrees) | 0.5 | 0.4 | 0.3 | 0.2 | 0.1 | 0.0 | −0.1 | −0.2 |
| 1 | −0.50 | 0.00 | 0 | 0.0 | ∘ | ∘ | ∘ | ∘ | ∘ | Δ | Δ | Δ |
| 2 | −0.50 | 0.00 | 0 | 90.0 | ∘ | ∘ | ∘ | ∘ | ∘ | Δ | Δ | Δ |
| 3 | −0.50 | 0.00 | 0 | 45.0 | ∘ | ∘ | ∘ | ∘ | ∘ | Δ | Δ | Δ |
| 4 | −0.50 | 0.00 | 0 | 135.0 | ∘ | ∘ | ∘ | ∘ | ∘ | Δ | Δ | Δ |
| 5 | −0.75 | 0.00 | 0 | 22.5 | ∘ | ∘ | ∘ | ∘ | ∘ | Δ | Δ | Δ |
| 6 | −0.75 | 0.00 | 0 | 112.5 | ∘ | ∘ | ∘ | ∘ | x | ∘ | Δ | Δ |
| 7 | −0.75 | 0.00 | 0 | 67.5 | ∘ | ∘ | ∘ | ∘ | ∘ | ∘ | Δ | Δ |
| 8 | −0.75 | 0.00 | 0 | 157.5 | ∘ | ∘ | ∘ | ∘ | x | Δ | Δ | Δ |
| 9 | −0.50 | −0.50 | 145 | 0.0 | ∘ | ∘ | ∘ | ∘ | ∘ | ∘ | Δ | Δ |
| 10 | −0.50 | −0.50 | 145 | 90.0 | ∘ | ∘ | ∘ | ∘ | ∘ | ∘ | Δ | Δ |
| 11 | −0.50 | −0.50 | 145 | 45.0 | ∘ | ∘ | ∘ | ∘ | Δ | Δ | Δ | Δ |
| 12 | −0.50 | −0.50 | 145 | 135.0 | ∘ | ∘ | ∘ | ∘ | ∘ | Δ | Δ | Δ |
| 13 | −0.50 | −0.50 | 145 | 22.5 | ∘ | ∘ | ∘ | ∘ | ∘ | ∘ | Δ | Δ |
| 14 | −0.50 | −0.50 | 145 | 112.5 | ∘ | ∘ | ∘ | ∘ | ∘ | ∘ | Δ | Δ |
| 15 | −0.50 | −0.50 | 145 | 67.5 | ∘ | ∘ | ∘ | ∘ | ∘ | ∘ | Δ | Δ |
| 16 | −0.50 | −0.50 | 145 | 157.5 | ∘ | ∘ | ∘ | ∘ | ∘ | ∘ | Δ | Δ |

TABLE 1B

| Direction | Test | Estimation | | | | | | | | |
|---|---|---|---|---|---|---|---|---|---|---|
| of dioptric | dioptric | dioptric | Function g(θ, i, j) | | | | | | | |
| power (degrees) | power (D) | power (D) | 0.5 | 0.4 | 0.3 | 0.2 | 0.1 | 0.0. | −0.1 | −0.2 |
| 90.0 | −0.50 | −0.92 | 1.000 | 0.999 | 0.993 | 0.948 | 0.751 | 0.553 | 0.507 | 0.501 |
| 0.0 | −0.50 | −0.70 | 1.000 | 1.000 | 0.998 | 0.982 | 0.879 | 0.634 | 0.520 | 0.502 |
| 135.0 | −0.50 | −0.56 | 1.000 | 1.000 | 0.999 | 0.991 | 0.932 | 0.713 | 0.540 | 0.505 |
| 45.0 | −0.50 | −1.05 | 1.000 | 0.998 | 0.987 | 0.905 | 0.666 | 0.527 | 0.503 | 0.500 |
| 112.5 | −0.75 | −0.71 | 1.000 | 1.000 | 0.999 | 0.995 | 0.957 | 0.776 | 0.563 | 0.508 |
| 22.5 | −0.75 | −0.90 | 1.000 | 1.000 | 0.998 | 0.985 | 0.898 | 0.656 | 0.525 | 0.503 |
| 157.5 | −0.75 | −0.55 | 1.000 | 1.000 | 1.000 | 0.998 | 0.980 | 0.867 | 0.622 | 0.518 |
| 67.5 | −0.75 | −1.06 | 1.000 | 1.000 | 0.996 | 0.969 | 0.818 | 0.585 | 0.512 | 0.501 |
| 90.0 | −0.84 | −0.92 | 1.000 | 1.000 | 0.999 | 0.990 | 0.925 | 0.699 | 0.536 | 0.504 |
| 0.0 | −0.66 | −0.70 | 1.000 | 1.000 | 0.999 | 0.992 | 0.939 | 0.730 | 0.545 | 0.506 |
| 135.0 | −0.52 | −0.56 | 1.000 | 1.000 | 0.999 | 0.992 | 0.936 | 0.722 | 0.543 | 0.505 |
| 45.0 | −0.98 | −1.05 | 1.000 | 1.000 | 0.999 | 0.990 | 0.929 | 0.706 | 0.538 | 0.505 |
| 112.5 | −0.64 | −0.71 | 1.000 | 1.000 | 0.999 | 0.991 | 0.930 | 0.709 | 0.539 | 0.505 |
| 22.5 | −0.86 | −0.90 | 1.000 | 1.000 | 0.999 | 0.991 | 0.935 | 0.719 | 0.542 | 0.505 |
| 157.5 | −0.52 | −0.55 | 1.000 | 1.000 | 0.999 | 0.992 | 0.940 | 0.731 | 0.546 | 0.506 |
| 67.5 | −0.98 | −1.06 | 1.000 | 1.000 | 0.999 | 0.990 | 0.924 | 0.698 | 0.536 | 0.504 |

TABLE 1C

| | | | | | | | | Total of |
|---|---|---|---|---|---|---|---|---|
| Logarithmic likelihood | | | | | | | | logarithmic |
| 0.5 | 0.4 | 0.3 | 0.2 | 0.1 | 0.0 | −0.1 | −0.2 | likelihood |
| 0.000 | −0.001 | −0.007 | −0.053 | −0.286 | −0.699 | −0.693 | −0.693 | −2.432 |
| 0.000 | 0.000 | −0.002 | −0.018 | −0.130 | −0.730 | −0.694 | −0.693 | −2.668 |
| 0.000 | 0.000 | −0.001 | −0.009 | −0.071 | −0.793 | −0.696 | −0.693 | −2.263 |
| 0.000 | −0.002 | −0.014 | −0.100 | −0.407 | −0.695 | −0.693 | −0.693 | −2.603 |
| 0.000 | 0.000 | −0.001 | −0.005 | −0.044 | −0.876 | −0.701 | −0.693 | −2.320 |
| 0.000 | 0.000 | −0.002 | −0.015 | −2.278 | −0.422 | −0.694 | −0.693 | −4.104 |
| 0.000 | 0.000 | 0.000 | −0.002 | −0.021 | −0.143 | −0.724 | −0.694 | −1.584 |
| 0.000 | 0.000 | −0.004 | −0.032 | −1.703 | −0.708 | −0.693 | −0.693 | −3.834 |
| 0.000 | 0.000 | −0.001 | −0.010 | −0.078 | −0.358 | −0.696 | −0.693 | −1.837 |
| 0.000 | 0.000 | −0.001 | −0.008 | −0.062 | −0.315 | −0.697 | −0.693 | −1.777 |
| 0.000 | 0.000 | −0.001 | −0.008 | −1.409 | −0.803 | −0.697 | −0.693 | −3.612 |
| 0.000 | 0.000 | −0.001 | −0.010 | −0.074 | −0.786 | −0.696 | −0.693 | −2.261 |
| 0.000 | 0.000 | −0.001 | −0.009 | −0.073 | −0.344 | −0.696 | −0.693 | −1.816 |
| 0.000 | 0.000 | −0.001 | −0.009 | −0.067 | −0.330 | −0.697 | −0.693 | −1.797 |
| 0.000 | 0.000 | −0.001 | −0.008 | −0.062 | −0.314 | −0.697 | −0.693 | −1.775 |
| 0.000 | 0.000 | −0.001 | −0.010 | −0.079 | −0.360 | −0.696 | −0.693 | −1.839 |

TABLE 2

| SC axis | | | | | | | | |
|---|---|---|---|---|---|---|---|---|
| S | C | | | | | | | Sum of |
| dioptric | dioptric | | JCC | | | Parameter | | logarithmic |
| power | power | Axis | mdp | J00 | J45 | a | b | likelihood |
| −0.54 | −0.54 | 147 | −0.81 | 0.11 | −0.25 | 5.14 | 21.46 | −38.123 |

TABLE 3

| | S | C | | | | | | | |
|---|---|---|---|---|---|---|---|---|---|
| | dioptric | dioptric | | | | | | | |
| | power | power | Axis | mdp | J00 | J45 | a | b | |
| Four times | −0.56 | 0.00 | 0 | −0.56 | 0.00 | 0.00 | 10.00 | 20.00 | Fix |
| estimation | | | | | | | | | |
| Eight times | −0.57 | −0.47 | 143 | −0.80 | 0.06 | −0.22 | 10.00 | 20.00 | Fix |
| estimation | | | | | | | | | |
| Twelve times | −0.60 | −0.40 | 145 | −0.80 | 0.07 | −0.19 | 10.00 | 20.00 | Fix |
| estimation | | | | | | | | | |
| Sixteen times | −0.55 | −0.41 | 146 | −0.75 | 0.08 | −0.19 | 10.00 | 20.00 | Fix |
| estimation | | | | | | | | | |
| Sixteen times | −0.54 | −0.54 | 147 | −0.81 | 0.11 | −0.25 | 5.14 | 21.46 | Simultaneous |
| estimation | | | | | | | | | estimation |

Also, although the subject is a myopic person in the aforementioned example, Table 4 and Table 5 mentioned below show results obtained by performing an eyesight test to which a hyperopic person is subjected. The halfway contents of calculation results are omitted, and only test conditions, response results in the eyesight test, and final estimation results are shown. This is an example in which the test was performed with the same trial lens from beginning to end. Estimation in the halfway process of four times and of twelve times is omitted. In this example, the test is performed while using a trial lens having the same dioptric power from one time to eight times and while using a trial lens having a different dioptric power from nine times to sixteen times.

In this example, without daring to perform the test up to sixteen times, the test may be ended with eight times although the accuracy becomes low. In that case, the subject wears the same lens in all the tests up to eight times. If the dioptric power of the lens is not changed in the halfway process, the subject may wear, for example, eyeglasses that the subject is now wearing, and the test may be performed while regarding it as a test lens. If so, it is possible to perform the test very conveniently because there is no need to use a test lens separately. Of course, the test may be performed up to twelve times or up to sixteen times while using a trial lens in the halfway process (while changing the lens), and, in that case, the test becomes more advantageous in accuracy. Furthermore, if the subject is not wearing eyeglasses at the present time, the test may be performed while wearing a plain trial lens that has no dioptric power. Also, the test may be performed in a naked-eye state without wearing such a plain trial lens that has no dioptric power.

TABLE 4

| | SC axis | | | Direction | | | | | | | | |
|---|---|---|---|---|---|---|---|---|---|---|---|---|
| | S | C | | of | | | | | | | | |
| Times | dioptric | dioptric | | optotype | LogMAR eyesight | | | | | | | |
| of test | power | power | Axis | (degrees) | 0.5 | 0.4 | 0.3 | 0.2 | 0.1 | 0.0 | −0.1 | −0.2 |
| 1 | 2.50 | 0.00 | 0 | 0.0 | ∘ | ∘ | ∘ | ∘ | x | Δ | ∘ | Δ |
| 2 | 2.50 | 0.00 | 0 | 90.0 | ∘ | ∘ | ∘ | ∘ | ∘ | ∘ | Δ | Δ |
| 3 | 2.50 | 0.00 | 0 | 45.0 | ∘ | ∘ | ∘ | ∘ | ∘ | ∘ | Δ | Δ |
| 4 | 2.50 | 0.00 | 0 | 135.0 | ∘ | ∘ | ∘ | ∘ | ∘ | ∘ | ∘ | Δ |
| 5 | 2.50 | 0.00 | 0 | 22.5 | ∘ | ∘ | ∘ | ∘ | ∘ | ∘ | ∘ | ∘ |
| 6 | 2.50 | 0.00 | 0 | 112.5 | ∘ | ∘ | ∘ | ∘ | ∘ | ∘ | ∘ | Δ |
| 7 | 2.50 | 0.00 | 0 | 67.5 | ∘ | ∘ | ∘ | ∘ | ∘ | ∘ | Δ | Δ |
| 8 | 2.50 | 0.00 | 0 | 157.5 | ∘ | ∘ | ∘ | ∘ | ∘ | Δ | Δ | Δ |
| 9 | 2.25 | 0.50 | 95 | 0.0 | ∘ | ∘ | ∘ | ∘ | ∘ | ∘ | ∘ | Δ |
| 10 | 2.25 | 0.50 | 95 | 90.0 | ∘ | ∘ | ∘ | ∘ | ∘ | ∘ | Δ | Δ |
| 11 | 2.25 | 0.50 | 95 | 45.0 | ∘ | ∘ | ∘ | Δ | ∘ | Δ | Δ | Δ |
| 12 | 2.25 | 0.50 | 95 | 135.0 | ∘ | ∘ | ∘ | ∘ | ∘ | ∘ | ∘ | Δ |
| 13 | 2.25 | 0.50 | 95 | 22.5 | ∘ | ∘ | ∘ | ∘ | ∘ | ∘ | Δ | Δ |
| 14 | 2.25 | 0.50 | 95 | 112.5 | ∘ | ∘ | ∘ | Δ | ∘ | Δ | Δ | Δ |
| 15 | 2.25 | 0.50 | 95 | 67.5 | ∘ | ∘ | ∘ | ∘ | ∘ | ∘ | Δ | Δ |
| 16 | 2.25 | 0.50 | 95 | 157.5 | ∘ | ∘ | ∘ | ∘ | ∘ | x | Δ | Δ |

TABLE 5

| | S | C | | | | | | | |
|---|---|---|---|---|---|---|---|---|---|
| | dioptric | dioptric | | | | | | | |
| | power | power | Axis | mdp | J00 | J45 | a | b | |
| Four times | | | | | | | 10.00 | 20.00 | Fix |
| estimation | | | | | | | | | |
| Eight times | 2.33 | 0.47 | 97 | 2.57 | 0.23 | 0.05 | 10.00 | 20.00 | Fix |
| estimation | | | | | | | | | |
| Twelve times | | | | | | | 10.00 | 20.00 | Fix |
| estimation | | | | | | | | | |
| Sixteen times | 2.30 | 0.44 | 94 | 2.52 | 0.22 | 0.03 | 10.00 | 20.00 | Fix |
| estimation | | | | | | | | | |
| Sixteen times | 2.35 | 0.41 | 95 | 2.55 | 0.20 | 0.03 | 7.47 | 14.09 | Simultaneous |
| estimation | | | | | | | | | estimation |

The aforementioned embodiment has been described merely as an example of a concrete embodiment to show the principle and concept of the present invention. In other words, the present invention is not limited to the aforementioned embodiment. The present invention can also be embodied in, for example, modes changed as follows.
Although decimal eyesight is used in “b. Determination of Startup Dioptric Power” of “A. Concerning Data Acquisition in Eyesight Test” of the aforementioned embodiment, an optotype (Landolt ring) displayed by log MAR may also be used in this stage (all log MAR). On the contrary, the eyesight test may be performed by use of decimal eyesight in all cases.
Optotypes other than the Landolt ring may be used.
Although the eyesight test is performed while displaying Landolt rings that are a set of 0 degrees in the right and 180 degrees in the left in “c. First Test and Acquisition of First Dioptric Power,” Landolt rings that are a pair of Landolt rings having mutually different directions by 180 degrees, such as a set of the upper and the lower or a set of the upper-right and the lower-left may be used. The direction of the Landolt ring is selected essentially randomly so as to be either one of two directions.
Either one of “c. First Test and Acquisition of First Dioptric Power” and “d. Second Test and Acquisition of Second Dioptric Power” may be omitted, and the step of “f. Third Test” may be performed immediately subsequent to the step of “b. Determination of Startup Dioptric Power” completely without performing both of “c. First Test and Acquisition of First Dioptric Power” and “d. Second Test and Acquisition of Second Dioptric Power.”
Although the eyesight test of each of the sixteen directions is performed one time in the aforementioned embodiment, the eyesight test thereof may be performed two or more times. Also, without performing the eyesight test evenly the same times with respect to all directions, the eyesight test may be repeatedly performed randomly in some directions.
Although an example in which the eyesight test is performed concerning the Landolt rings that face sixteen directions has been described in the aforementioned embodiment, the eyesight test may be performed concerning the Landolt rings that face sixteen or less directions. On the contrary, if the number of the directions is more than sixteen, the accuracy of estimation is not necessarily improved, and the labor hour for the test will be increased. Also, if the number of the directions is too many, a subject cannot specify the direction of an optotype (or cannot easily specify its direction), and there is a concern that errors will occur in the test.

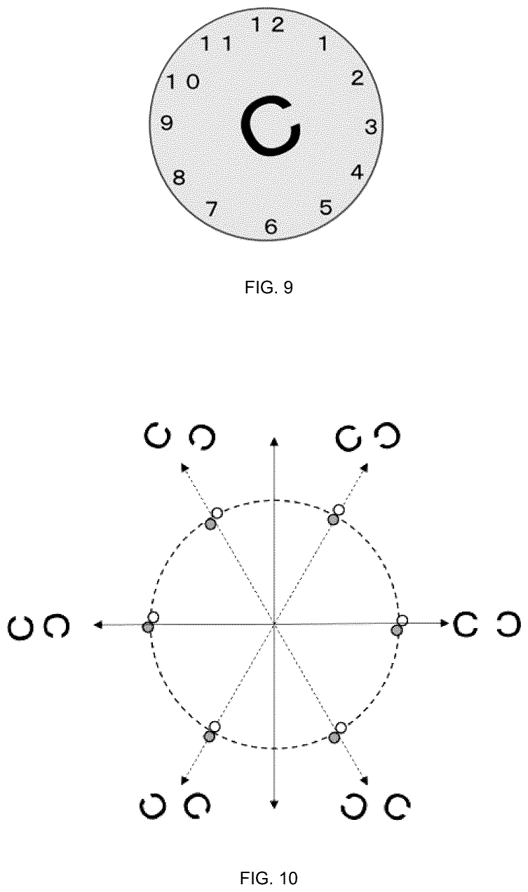

For example, an eyesight chart having optotypes (Landolt rings) of twelve directions may be used, and a subject may be asked to answer the direction of a Landolt ring by means of numerals of a clock face as in FIG. 9. The reason is that, in the clock face, the numerals are placed at positions that are divided at twelve equal intervals just equiangularly (with 30-degree steps). This is inferior in accuracy to a method in which the eyesight test is performed in sixteen directions. However, the balance in which the labor hour and the accuracy are required depends on an operator, and therefore this clock face method is also useful.

In this case, the process from “b. Determination of Startup Dioptric Power” to the determination of the third dioptric power in “f. Third Test” is the same as the method in which the test is performed in sixteen directions. Landolt rings in the directions of 2-8 hours and 11-5 hours are used in the third test, and Landolt rings in the directions of 1-7 hours and 10-4 hours are used in the fourth test. The direction of two Landolt rings facing each other by 180 degrees and the direction of 12 are shown in FIG. 10. The calculation based on acquired data is the same as the calculation of “B. Dioptric-Power Estimation Method” mentioned above.

Table 6 and Table 7 show results obtained by performing the eyesight test by use of an eyesight chart of Landolt rings having twelve directions as described above. The halfway contents of calculation results are omitted, and only test conditions, response results in the eyesight test, and final estimation results are shown. This is an example in which an eyesight chart having Landolt rings facing twelve directions as in FIG. 10 is used and in which the test with a single trial lens is performed six times, and the test with three different trial lenses is performed.

TABLE 6

| | SC axis | | | Direction | | | | | | | | |
|---|---|---|---|---|---|---|---|---|---|---|---|---|
| | S | C | | of | | | | | | | | |
| Times | dioptric | dioptric | | optotype | LogMAR eyesight | | | | | | | |
| of test | power | power | Axis | (degrees) | 0.5 | 0.4 | 0.3 | 0.2 | 0.1 | 0.0 | −0.1 | −0.2 |
| 1 | −5.25 | 0.00 | 0 | 0.0 | ∘ | ∘ | ∘ | ∘ | ∘ | ∘ | Δ | Δ |
| 2 | −5.25 | 0.00 | 0 | 30.0 | ∘ | ∘ | ∘ | ∘ | ∘ | Δ | Δ | Δ |
| 3 | −5.25 | 0.00 | 0 | 60.0 | ∘ | ∘ | ∘ | ∘ | ∘ | ∘ | Δ | Δ |
| 4 | −5.25 | 0.00 | 0 | 90.0 | ∘ | ∘ | ∘ | ∘ | ∘ | ∘ | x | Δ |
| 5 | −5.25 | 0.00 | 0 | 120.0 | ∘ | ∘ | ∘ | ∘ | ∘ | ∘ | Δ | Δ |
| 6 | −5.25 | 0.00 | 0 | 150.0 | ∘ | ∘ | ∘ | ∘ | ∘ | ∘ | ∘ | Δ |
| 7 | −5.25 | −0.25 | 55 | 0.0 | ∘ | ∘ | ∘ | ∘ | ∘ | ∘ | Δ | Δ |
| 8 | −5.25 | −0.25 | 55 | 30.0 | ∘ | ∘ | ∘ | ∘ | ∘ | ∘ | Δ | Δ |
| 9 | −5.25 | −0.25 | 55 | 60.0 | ∘ | ∘ | ∘ | ∘ | ∘ | ∘ | ∘ | Δ |
| 10 | −5.25 | −0.25 | 55 | 90.0 | ∘ | ∘ | ∘ | ∘ | ∘ | ∘ | ∘ | Δ |
| 11 | −5.25 | −0.25 | 55 | 120.0 | ∘ | ∘ | ∘ | ∘ | ∘ | ∘ | ∘ | Δ |
| 12 | −5.25 | −0.25 | 55 | 150.0 | ∘ | ∘ | ∘ | ∘ | ∘ | x | Δ | Δ |
| 13 | −5.00 | −0.25 | 15 | 0.0 | ∘ | ∘ | ∘ | x | ∘ | ∘ | Δ | Δ |
| 14 | −5.00 | −0.25 | 15 | 30.0 | ∘ | ∘ | ∘ | ∘ | ∘ | Δ | Δ | Δ |
| 15 | −5.00 | −0.25 | 15 | 60.0 | ∘ | ∘ | ∘ | ∘ | ∘ | ∘ | ∘ | Δ |
| 16 | −5.00 | −0.25 | 15 | 90.0 | ∘ | ∘ | ∘ | ∘ | x | x | Δ | Δ |
| 17 | −5.00 | −0.25 | 15 | 120.0 | ∘ | ∘ | ∘ | ∘ | ∘ | Δ | Δ | Δ |
| 18 | −5.00 | −0.25 | 15 | 150.0 | ∘ | ∘ | ∘ | ∘ | ∘ | Δ | Δ | Δ |

TABLE 7

| | S | C | | | | | | | |
|---|---|---|---|---|---|---|---|---|---|
| | dioptric | dioptric | | | | | | | |
| | power | power | Axis | mdp | J00 | J45 | a | b | |
| Six times | −5.00 | −0.26 | 53 | −5.13 | −0.04 | 0.12 | 10.00 | 10.00 | Fix |
| estimation | | | | | | | | | |
| Twelve times | −5.10 | −0.16 | 13 | −5.18 | 0.07 | 0.03 | 10.00 | 10.00 | Fix |
| estimation | | | | | | | | | |
| Eighteen times | −5.14 | −0.17 | 8 | −5.23 | 0.08 | 0.02 | 10.00 | 10.00 | Fix |
| estimation | | | | | | | | | |
| | | | | | | | 10.00 | 10.00 | Fix |
| Eighteen times | −5.15 | −0.17 | 14 | −5.24 | 0.07 | 0.04 | 15.25 | 21.69 | Simultaneous |
| estimation | | | | | | | | | estimation |

Also, the eyesight test may be performed in eight directions. Although this is inferior in accuracy to the method of the test in the sixteen/twelve directions, the labor hour is small, and therefore there is a practical advantage in consideration of balance. In this case, following the method of the eyesight test in sixteen directions, the test is realized by using Landolt rings up to the 45-degree step without using a Landolt ring of the 22.5-degree step. In the same way as in the aforementioned method, the third test is performed, and the fourth dioptric power is estimated, and, thereafter, the test using the Landolt rings in eight directions and the estimation of a dioptric power are repeatedly performed.
The eyesight test may be performed in six directions. Although this is inferior in accuracy to the aforementioned method, there is an effect by which the labor hour is minimized. This is a method in which the test is performed in twelve directions, and is realized by using optotypes (Landolt rings) up to the 60-degree step without using a Landolt ring of the 30-degree step.

1. A dioptric power determination method for determining a dioptric power of an ophthalmic lens when eyesight is corrected by the ophthalmic lens, the method comprising the steps of:
setting a target value of the eyesight in a state in which a subject has been subjected to refractive-error correction by the ophthalmic lens;
allowing the subject to watch a plurality of optotypes facing various different directions in a state of wearing a test lens or in a naked-eye state while regarding the target value as target eyesight;
asking the subject to answer the direction of the optotype;
if a result in which a correct answer and an incorrect answer, or a correct answer and an indistinguishable answer, or a correct answer, an incorrect answer, and an indistinguishable answer coexist is obtained, estimating a dioptric power by which the subject visually perceives the optotype with a fixed predetermined probability in all directions in a circumferential direction of the optotype corresponding to the target eyesight on a basis of a relationship between a response and a dioptric power corresponding to the response; and
determining the dioptric power of the ophthalmic lens of the subject on a basis of an estimation result obtained by estimating the dioptric power.

2. The dioptric power determination method according to claim 1, wherein the dioptric power of the test lens is changed, and the subject is allowed to wear the test lens, and the subject is allowed to repeatedly watch the optotype in order to mix a correct answer and an incorrect answer, or a correct answer and an indistinguishable answer, or a correct answer, an incorrect answer, and an indistinguishable answer together when the subject is allowed to watch the optotype in a state in which the subject is wearing the test lens.

3. The dioptric power determination method according to claim 1, wherein when the subject is allowed to watch the optotype in a state in which the subject is wearing the test lens, a dioptric power of the test lens to mix a correct answer and an incorrect answer, or mix a correct answer and an indistinguishable answer together, or mix a correct answer, an incorrect answer, and an indistinguishable answer together is set at a dioptric power of an eyeglass lens regularly used by the subject or at a dioptric power close to the dioptric power of the eyeglass lens.

4. The dioptric power determination method according to claim 3, wherein the test lens has a same dioptric power in each watching and in each answering, and the subject is allowed to wear this test lens and is allowed to repeatedly watch the optotype.

5. The dioptric power determination method according to claim 2, wherein when the subject is allowed to wear the test lens, the subject wears the test lens having a dioptric power differing in accordance with a test situation, and repeatedly watches the optotype.

6. The dioptric power determination method according to claim 2, wherein the optotype watched by the subject is a plurality of optotypes that differ in size including the optotype corresponding to the target eyesight.

7. The dioptric power determination method according to claim 2, wherein the optotype is displayed by an eyesight-test chart so that different-sized optotypes are visually perceivable at a glance.

8. The dioptric power determination method according to claim 7, wherein a direction of the optotype in a group of the optotypes displayed on the eyesight-test chart consists of two kinds of directions, i.e., a certain direction and a direction 180 degrees opposite to the certain direction.

9. The dioptric power determination method according to claim 2, wherein a number of kinds of the directions of the optotype is six to sixteen.

10. The dioptric power determination method according to claim 2, wherein an eyesight value according to a size of the optotype is a log MAR form.

11. The dioptric power determination method according to claim 2, wherein the optotype is a Landolt ring.

12. The dioptric power determination method according to claim 2, wherein the estimation is performed by optimization calculation according to a maximum-likelihood method.

13. The dioptric power determination method according to claim 12, wherein calculation by which a likelihood is calculated in the optimization calculation is performed by logistic regression, and is estimated on a basis of the likelihood.

14. The dioptric power determination method according to claim 3, wherein the optotype watched by the subject is a plurality of optotypes that differ in size including the optotype corresponding to the target eyesight.

15. The dioptric power determination method according to claim 3, wherein the optotype is displayed by an eyesight-test chart so that different-sized optotypes are visually perceivable at a glance.

16. The dioptric power determination method according to claim 15, wherein a direction of the optotype in a group of the optotypes displayed on the eyesight-test chart consists of two kinds of directions, i.e., a certain direction and a direction 180 degrees opposite to the certain direction.

17. The dioptric power determination method according to claim 3, wherein a number of kinds of the directions of the optotype is six to sixteen.

18. The dioptric power determination method according to claim 3, wherein an eyesight value according to a size of the optotype is a log MAR form.

19. The dioptric power determination method according to claim 3, wherein the optotype is a Landolt ring.

20. The dioptric power determination method according to claim 3, wherein the estimation is performed by optimization calculation according to a maximum-likelihood method.
